(19) United States
(12) Patent Application Publication
(10) Pub. No.: US 2017/0087002 A1
(43) Pub. Date: Mar. 30, 2017

(54) PRESSURE RELIEF PAD FOR THE PREVENTION OF PRESSURE ULCERS

(71) Applicant: TAICEND TECHNOLOGY CO.,LTD, Kaohsiung City (TW)

(72) Inventors: GOU-DON CHU, KAOHSIUNG CITY (TW); KUN-CHENG TSAI, KAOHSIUNG CITY (TW)

(21) Appl. No.: 15/267,741

(22) Filed: Sep. 16, 2016

(30) Foreign Application Priority Data
Sep. 30, 2015 (TW) 104132286

(51) Int. Cl. A61F 5/30; A61M 16/06

(52) U.S. Cl. CPC A61F 5/30; A61M 16/0605

(57) ABSTRACT

A pressure relief pad for the prevention of pressure ulcers includes a pressure relief layer that is a foam structure that evenly disperses forces that the skin is subjected to; a thin film layer that is a hydrophilic thin film structure that attaches to the skin; and a release liner having a smooth surface and a rough surface, and including a predetermined pattern. The pressure relief layer, the thin film layer and the release liner are attached to and disposed on each other as a stack in that order, and have a same shape. The pressure relief pad may reduce pressure and shearing forces that the skin is subjected to, in order to prevent pressure ulcers from occurring.

### CROSS-REFERENCE TO RELATED APPLICATION

This application claims priority from Taiwan Patent Application No. 104132286, filed on Sep. 30, 2015, in the Taiwan Intellectual Property Office, the content of which is hereby incorporated by reference in its entirety for all purposes.

### BACKGROUND OF THE INVENTION

1. Field of the Invention

The present invention relates to a pressure relief pad, and more particularly to a pressure relief pad that prevents pressure ulcers.

2. Description of the Related Art

Pressure sores, also known as bed sores, mainly occur due to prolonged and continuous pressure or compression on the skin that covers bony areas of the body, resulting in partial damage to skin and the subcutaneous tissue. Pressure sores can be described to be in one of four stages—from mild skin ulceration in the initial stage to full-thickness tissue loss in stage four. In severe cases, the bones and tendons are exposed, causing osteomyelitis. Pressure sores are commonly seen in those who are bedridden, confined to a wheelchair, have impaired mobility, require prolonged use of noninvasive positive pressure ventilator (NPPV), or those with diabetes. Such patients usually have to maintain a certain posture over a long period of time, where the contact and leaning against physical objects (e.g. the mattress, bedding, a ventilator mask or any auxiliary medical tubing) of the skin at the body's bony prominences can cause shearing forces, pressure and friction, leading to discomfort and pressure sores.

For patients on NPPV, a nasal or full face mask is closely fitted to the face so that a seal is formed between the face and the mask without leakage and thus ensure effective ventilation support. However, the prolonged use of NPPV can be uncomfortable and can often lead to facial pressure sores. Currently, the commonly used cushioning component or buffer between the mask and the patient's skin is a hydrocolloid dressing (artificial skin). A hydrocolloid dressing is a skin-friendly hydrocolloid-based dressing. Although such skin-friendly dressings can prevent direct contact between the mask and the skin, its relative thinness has a limited effect on pressure reduction. The removal of the hydrocolloid dressing also often causes problems such as skin tearing, pain and residual fragments of dressing left on the skin.

For effective NPPV, the mask must have a close fit to the face and so the buffer must conform to both the face contours and the mask. At present, hydrocolloid dressings are commercially available in single square sheets of approximately 10 cm by 10 cm and must be trimmed according to the contours of each patient's face. The trimming process is also time and labor-consuming as it involves repetitive outlining and shaping of the square sheets, and mistakes in this process could mean wasting of medical resources.

### SUMMARY OF THE INVENTION

In view of the limitations of the prior art, the primary object of the present invention is to provide a pressure relief pad to prevent pressure ulcers. The pressure relief pad combines a layer of a fine foam material with a layer of a hydrophilic thin film to reduce pressure, friction and shearing forces and so keep the skin or a wound on the skin comfortable and prevent pressure ulcers.

In order to achieve the above objects, the present invention provides a pressure relief pad that prevents pressure ulcers. The pressure relief pad of the present invention includes a pressure relief layer, a thin film layer, and a release liner. The pressure relief layer is a cellular foam structure that evenly disperses forces that the skin is subjected to. The thin film layer is a hydrophilic thin film structure for attachment to the skin. The thin film layer includes a first surface and a second surface. One surface of the pressure relief layer is in contact with the second surface of the thin film layer, rendering the pressure relief layer to be disposed on the thin film layer. The release liner includes a smooth surface and a rough surface. The first surface of the thin film layer is in contact with the smooth surface of the release liner, rendering the thin film layer to be disposed on the release liner. The rough surface includes a predetermined pattern. The pressure relief layer, the thin film layer and the release liner have a same shape.

Preferably, the pressure relief layer of the pressure relief pad of the present invention includes polyurethane elastomers.

Preferably, the foam structure of the pressure relief layer has a density of 10-50 kg/m³.

Preferably, the thin film layer of the pressure relief pad of the present invention includes polyurethane (PU).

Preferably, the proportion of a thickness of the pressure relief layer to a thickness of the thin film layer of the present invention is in the range of 68:1 to 500:1.

Preferably, the coefficient of restitution of the pressure relief pad is less than 10% according to a standardized test for flexible cellular polymeric materials.

Preferably, the pressure relief pad has a moisture vapor transmission rate of greater than 2000 g/m² each day.

In order to achieve the above objects, the present invention further provides a pressure relief pad to prevent pressure ulcers on the face. The pressure relief pad includes a pressure relief layer, a thin film layer, and a release liner. The pressure relief layer is a cellular foam structure. The thin film layer is a hydrophilic thin film structure. The pressure relief layer is in contact with a surface of the thin film layer, rendering the pressure relief layer to be disposed on the thin film layer. The release liner includes a smooth surface and a rough surface. Another surface of the thin film layer is in contact with the smooth surface of the release liner, rendering the thin film layer to be disposed on the release liner. The pressure relief layer, the thin film layer and the release liner have a same shape. The rough surface includes a bottle-shaped pattern. The bottle-shaped pattern includes a bottleneck portion, a bottle body portion, and a cutout portion. The bottleneck portion protrudes outward relative to the center of the pressure relief pad. The bottle body portion extends from the bottleneck portion toward two sides thereof. The widest part of the bottle body portion is about 3-5 times as wide as the bottleneck portion. The cutout portion is disposed at the center of the bottle body portion and aligned with the bottleneck portion.

Preferably, the bottleneck portion has a width of 30 mm to 40 mm.

Preferably, the cutout portion is cut and separated from the bottle body portion, so that the bottle body portion has a hollow portion. The bottleneck portion corresponds in position to the bridge of a user's nose. The bottle body portion covers the user's cheeks and philtrum and is attached tightly to the skin. The user's nose is exposed out of the hollow portion. The cutout portion is placed on the user's forehead and attached tightly to the skin.

Given these preferred properties, the pressure relief pad for the prevention of pressure ulcers of the present invention has one or more of the following advantages:

1. The pressure relief pad of the present invention combines the pressure relief layer with the thin film layer, both made of polyurethane, to reduce pressure, friction and shearing forces that the skin is subjected to due to contact with objects, so as to prevent pressure ulcers.

2. The pressure relief pad of the present invention uses the hydrophilic thin film layer to maintain contact with the skin. This skin contact layer does not strongly adhere to wounds with moisture that might be present on the skin, and so the pressure relief pad can be easily removed and replaced without causing any discomfort.

3. The pressure relief pad of the present invention uses a bottle-shaped pattern that closely fits the user's face. This is to keep the skin from drying, to reduce the pressure from the ventilator mask, and so effectively prevent pressure ulcers.

### BRIEF DESCRIPTION OF THE DRAWINGS

FIG. 1 is a perspective view of a pressure relief pad in accordance with a preferred embodiment of the present invention;

FIG. 2 is a plan view of a predetermined pattern of a pressure relief pad in accordance with a preferred embodiment of the present invention; and

FIG. 3 is a perspective view of the pressure relief pad in accordance with a preferred embodiment of the present invention when in use.

### DESCRIPTION OF THE PREFERRED EMBODIMENTS

The embodiments of the present invention will now be described, by way of example only, with reference to the accompanying drawings. The present invention, however, may be embodied in various different forms, and should not be construed as only being limited to the illustrated embodiments herein.

The present invention discloses a pressure relief pad that prevents pressure ulcers. The pressure relief pad combines a fine foam structure with a hydrophilic thin film structure, which can evenly disperse forces that the skin is subjected to, so as to prevent pressure ulcers.

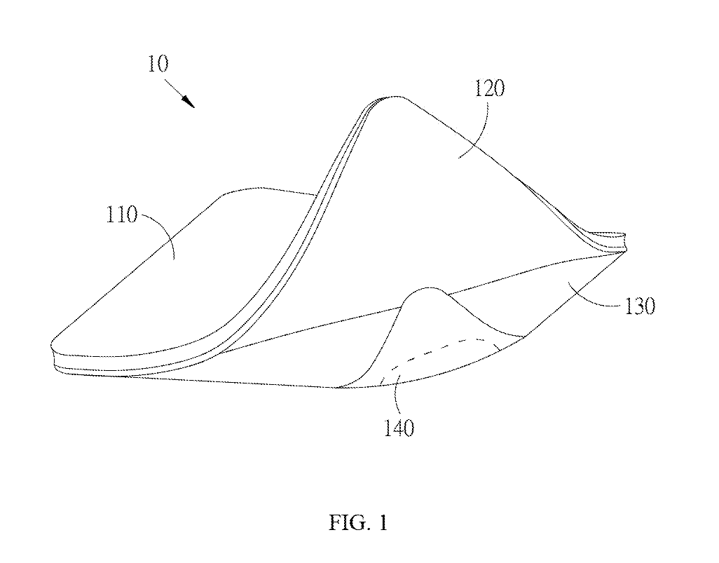

FIG. 1 is a perspective view of a pressure relief pad in accordance with a preferred embodiment of the present invention. As shown in FIG. 1, the pressure relief pad 10 of the embodiment includes a pressure relief layer 110, a thin film layer 120, a release liner 130, and a predetermined pattern 140.

The pressure relief layer 110 is a cellular foam structure, that evenly disperses forces that the skin is subjected to. The thin film layer 120 is a hydrophilic thin film structure for attachment to the skin. The thin film layer 120 includes a first surface and a second surface. One surface of the pressure relief layer 110 is in contact with the second surface of the thin film layer 120, such that the pressure relief layer 110 is disposed on the thin film layer 120. The pressure relief layer 110 and the thin film layer 120 may be bound with adhesive or directly adhered to each other. In the pressure relief pad 10 of the embodiment, the pressure relief layer 110 and the second surface of the thin film layer 120 are bound with polyurethane (PU) glue for medical use.

In the embodiment, the pressure relief layer 110 of the pressure relief pad 10 is made of polyurethane elastomers. The foam structure of the pressure relief layer 110 made of polyurethane elastomer has a density of 10-50 kg/m³. Preferably, the density is 38 kg/m³.

On the other hand, the main material of the thin film layer 120 of the pressure relief pad 10 of the embodiment is polyurethane (PU) and made from the precursors TDI/MDI/IPDI and PG/PTMG in a liquid-liquid reaction. The thin film layer 120 of the embodiment is hydrophilic, has a low viscosity and a low sensitivity. Due to the cellular nature of the PU material, the thin film layer 120 is also permeable to moisture.

The first surface of the thin film layer 120 is in contact with the release liner 130. The thin film layer 120 is attached to a smooth surface of the release liner 130 due to its hydrophilic nature rather than the use of an adhesive. Thus, the pressure relief layer, the thin film layer and the release liner are attached to one another to form a pressure relief pad having a thickness. The pressure relief layer has a thickness of 5 mm-20 mm. The thin film layer has a thickness of 0.01 mm-0.3 mm. The ratio of the thickness of the pressure relief layer to the thickness of the thin film layer is in the range of 68:1 to 500:1. In the embodiment, the thickness of the pressure relief layer is 10 mm. Preferably, the thickness of the thin film layer is 0.02 mm. Preferably, the ratio of the thickness of the pressure relief layer to the thickness of the thin film layer is 500 to 1. Furthermore, the pressure relief pad 10 of the embodiment of the present invention can be in any shape. But, the pressure relief layer 110, the thin film layer 120, and the release liner 130 must have a same shape. In the embodiment, the pressure relief pad is a 15 cm by 15 cm square.

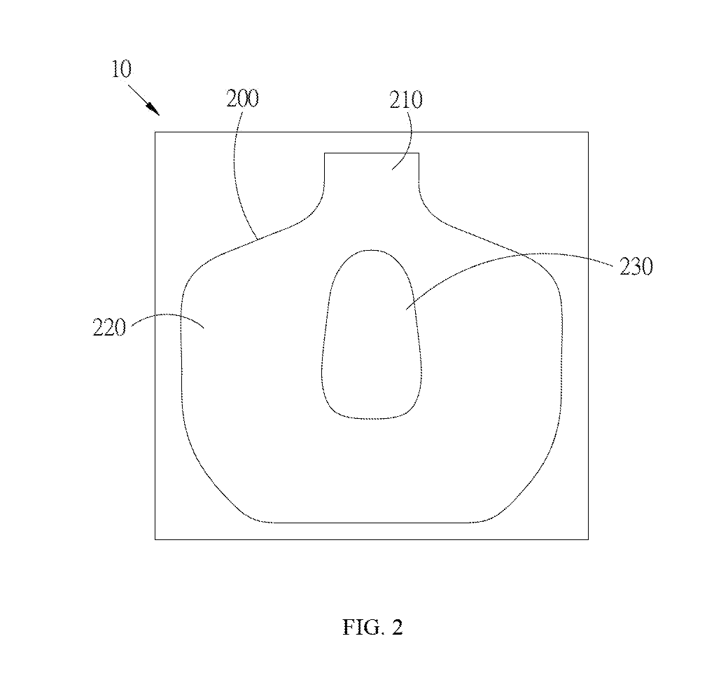

According to the pressure relief pad of the embodiment of the present invention, the predetermined pattern 140 is disposed on a rough surface of the release liner 130. The predetermined pattern 140 may be any desired pattern. To fit with the shape of the user's body, the predetermined pattern 140 is cut to a suitable shape. In the embodiment, the pressure relief pad is used together with a mask of a noninvasive positive pressure ventilator. The predetermined pattern 140 may be a bottle-shaped pattern, as shown in FIG. 2. The predetermined pattern 140 may be printed or depicted on the rough surface of the release liner 130 with a broken line or a solid line, or it may be cut in advance on the rough surface of the release liner 130 (the back of the pressure relief pad) by using a perforation cutter.

According to the pressure relief pad of the embodiment of the present invention, when the thickness of the pressure relief layer is 10 mm and the thickness of the thin film layer is 0.02 mm, the physical characteristics of the pressure relief pad are shown in the following table, Table 1.

TABLE 1
Physical characteristics of the pressure relief pad

| Physical characteristics | Value | Unit |
|---|---|---|
| Density | 38 ± 2 | kg/m³ |
| Hardness | 25 ± 5 | Asker ° F. |
| Tear strength | 0.7 | kg/cm |
| Tensile strength | 1.5 | kg/cm² |
| Elongation | 120 | % |
| Coefficient of restitution | 3 | % |
| (compression strength) | | |

To find out the reduction in permeability due to pressure of the pressure relief pad of the embodiment of the present invention, a functionality test is carried out on the pressure relief pad of the embodiment of the present invention.

The pressure relief pad of the embodiment was subjected to a compression test based on two international standardized testing processes. The inventor conducted a ball rebound test on the pressure relief pad based on the international standard process for molded polyurethane of a flexible cellular material established by the American Society for Testing and Materials (ASTM). The inventor also conducted a ball rebound test on the pressure relief pad based on the international standard process for flexible cellular materials established by the International Organization for Standardization (ISO). The test methods described in both international standards are common knowledge for a person skilled in the pertinent technical field and shall not be elaborated here. The standardized tests measure a rebound coefficient of less than 10% for the pressure relief pad of the embodiment. In the actual test, a ball was held in the air and then dropped down toward the pressure relief pad of the embodiment lying on the ground. There was no immediate rebound upon impact on the pressure relief pad of the present invention and a rebound coefficient of 3% was obtained. This result shows that the pressure relief pad of the embodiment of the present invention is effective in absorbing the pressure it is subjected to, dispersing the effect of the impact from the object and spreading the pressure evenly.

The inventor also conducted a permeability test on the pressure relief pad of the embodiment of the present invention in accordance with the standard test to measure the moisture vapor transmission rate of flexible cellular materials established by the ASTM. The test method described in the international standard is common knowledge for a person skilled in the pertinent technical field and shall not be elaborated here. The result of the test shows that the pressure relief pad of the embodiment of the present invention has a moisture vapor transmission rate greater than 2000 g/m² for each day. In other words, the thin film structure and cellular foam structure of the pressure relief pad of the present invention are satisfactorily permeable. When applied to the skin, the pressure relief pad will allow excess moisture present on the surface of the skin to evaporate away, thus maintaining appropriate levels of moisture.

FIG. 2 is a plan view of the predetermined pattern of the pressure relief pad in accordance with the preferred embodiment of the present invention. In the embodiment of the present invention, the predetermined pattern is a bottle-shaped pattern 200. The bottle-shaped pattern 200 includes a bottleneck portion 210, a bottle body portion 220, and a cutout portion 230. The bottleneck portion 210 protrudes outward relative to the center of the pressure relief pad 10. The bottleneck portion 210 has a width of 30 mm to 40 mm. In the embodiment, the width of the bottleneck portion 210 is 32 mm. The bottle body portion 220 extends from the bottleneck portion 210 toward two sides thereof. The widest portion of the bottle body portion 220 is about 3-5 times as wide as the bottleneck portion 210. In this embodiment, the widest portion of the bottle body portion 220 is about 4 times as wide as the bottleneck portion 210, namely 128 mm. The cutout portion 230 is disposed at the center of the bottle body portion 220 and aligned with the bottleneck portion 210. When the pressure relief pad is used, the cutout portion 230 will be cut to separate it from the bottle body portion 220, so that the bottle body portion 220 has a hollow portion. To be noted is that in the embodiment and in the drawings the cutout portion 230 has a nose shape that is suitable for use on the user's face with a ventilator mask. However, the shape and the area of the cutout portion are not limited thereto.

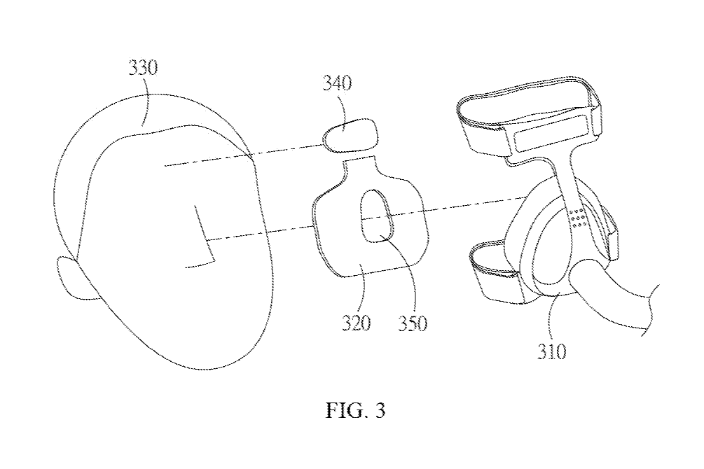

FIG. 3 is a perspective view of the pressure relief pad in accordance with the preferred embodiment of the present invention when in use. The pressure relief pad of the embodiment of the present invention may, for example, be used with a noninvasive positive pressure ventilator mask 310 on a patient 330 that necessitates noninvasive positive pressure ventilation. For this use, the pressure relief pad of the embodiment of the present invention may, for example, be used as follows. The entire pressure relief pad is taken out from its packaging. The packaging may be sterile packaging to reduce the risk of secondary infection. Before applying the pressure relief pad, the surface of the skin must be cleaned and dried. The pressure relief pad of the present invention is cut according to the desired shape and size. In the embodiment, the pressure relief pad is cut to the bottle-shaped pattern 320, as shown in FIG. 3. The central cutout portion is cut to have a nose shape and is separated from the bottle body portion. Thus, the bottle-shaped pattern 320 has a hollow portion 350. Next, the release liner of the pressure relief pad is peeled off. The thin film layer of the pressure relief pad is brought into contact with the skin, so that it covers the skin evenly. As shown in FIG. 3, the bottleneck portion of the pressure relief pad corresponds in position to the bridge of the patient's nose, and the bottle body portion of the pressure relief pad covers the patient's cheeks and philtrum. The bottle-shaped pattern 320 with hollow portion 350 is attached tightly to the skin. The patient's nose is exposed out of the hollow portion 350 of the bottle body portion. Furthermore, the separate cutout portion 340 is placed on the patient's forehead and is also attached tightly to the skin. Then, the patient may wear the noninvasive positive pressure ventilator mask 310. The noninvasive positive pressure ventilator mask 310 is brought into contact with the pressure relief layer to complete the placement of the pressure relief pad.

In a practical implementation, in which the pressure relief pad of the embodiment of the present invention is applied to the skin, a test has shown that shearing forces can be reduced by 50% compared to wearing the ventilator mask only without any extra components.

The pressure relief layer of the pressure relief pad of the present invention is made of polyurethane elastomers to form a high-density fine foam structure. The present invention provides greater pressure relief than that of the prior art. The pressure relief pad is able to evenly disperse and absorb pressure and shearing forces caused by contact with an external object, thus reducing the pressure and shearing forces that the skin is subjected to, and so effectively preventing pressure ulcers.

The pressure relief pad of the present invention uses the hydrophilic thin film layer to maintain contact with the skin. This skin contact layer does not adhere strongly to wounds with moisture that might be present on the skin, and so the pressure relief pad can be easily removed and replaced without causing any discomfort. The hydrophilic thin film layer can keep the skin around the wound dry and block any wound exudate from flowing to the surrounding skin and so reduce the risk of moistening the wound.

The pressure relief pad is not limited to be used on its own. In further embodiments, the pressure relief pad of the present invention can be used together with ventilative tape or waterproof transparent adhesive film. After the pressure relief pad is placed on the skin, some ventilative tape may be used to secure the pressure relief pad in place, or the outmost layer may be covered with the waterproof transparent adhesive film to shield the area from water when washing for hygiene.

To summarize, the pressure relief pad of the present invention uses a dual-layer structure composed of the pressure relief layer and the thin film layer to effectively reduce the pressure, compressive stresses and shearing forces that the skin may be subjected to due to contact with an external object, and so prevent pressure ulcers. The pressure relief pad of the present invention uses the hydrophilic thin film layer to maintain contact with the skin and so reduce the friction of the pressure relief pad against the skin. The pressure relief pad of the present invention does not strongly adhere to wounds that might be present on the skin and so can be easily removed and replaced without causing any discomfort.

This disclosure has been described by means of specific embodiments. However, numerous modifications and variations can be made thereto by those skilled in the art without departing from the scope and spirit of this disclosure set forth in the claims.

What is claimed is:

1. A pressure relief pad preventing pressure ulcers, comprising:
a pressure relief layer, the pressure relief layer being a cellular foam structure used to evenly disperses forces that the skin is subjected to;
a thin film layer, the thin film layer being a hydrophilic thin film structure that attaches to the skin, the thin film layer comprising a first surface and a second surface, one surface of the pressure relief layer being in contact with the second surface of the thin film layer, rendering the pressure relief layer to be disposed on the thin film layer; and
a release liner, the release liner comprising a smooth surface and a rough surface, the first surface of the thin film layer being in contact with the smooth surface of the release liner, rendering the thin film layer to be disposed on the release liner, the rough surface comprising a predetermined pattern,
wherein, the pressure relief layer, the thin film layer and the release liner have a same shape.

2. The pressure relief pad preventing pressure ulcers as claimed in claim 1, wherein the pressure relief layer comprises polyurethane elastomers.

3. The pressure relief pad preventing pressure ulcers as claimed in claim 2, wherein the foam structure of the pressure relief layer has a density of 10-50 kg/m³.

4. The pressure relief pad preventing pressure ulcers as claimed in claim 1, wherein the thin film layer comprises polyurethane (PU).

5. The pressure relief pad preventing pressure ulcers as claimed in claim 1, wherein the proportion of a thickness of the pressure relief layer to a thickness of the thin film layer is in the range of 68:1 to 500:1.

6. The pressure relief pad preventing pressure ulcers as claimed in claim 1, wherein the coefficient of restitution of the pressure relief pad is less than 10% according to a standardized test for flexible cellular polymeric materials.

7. The pressure relief pad preventing pressure ulcers as claimed in claim 1, wherein the pressure relief pad has a moisture vapor transmission rate greater than 2000g/m²for each day.

8. A pressure relief pad preventing facial pressure ulcers, comprising:
a pressure relief layer, the pressure relief layer being a cellular foam structure;
a thin film layer, the thin film layer being a hydrophilic thin film structure, the pressure relief layer being in contact with a surface of the thin film layer, rendering the pressure relief layer to be disposed on the thin film layer; and
a release liner, the release liner comprising a smooth surface and a rough surface, another surface of the thin film layer being in contact with the smooth surface of the release liner, rendering the thin film layer to be disposed on the release liner, furthermore the pressure relief layer, the thin film layer and the release liner having a same shape;
wherein the rough surface comprises a bottle-shaped pattern, and the bottle-shaped pattern comprises:
a bottleneck portion, the bottleneck portion protruding outward relative to a center of the pressure relief pad;
a bottle body portion, the bottle body portion extending from the bottleneck portion toward two sides thereof, a widest portion of the bottle body portion being about 3-5 times as wide as the bottleneck portion; and
a cutout portion, the cutout portion being disposed at a center of the bottle body portion and aligned with the bottleneck portion.

9. The pressure relief pad preventing facial pressure ulcers as claimed in claim 8, wherein the bottleneck portion has a width of 30 mm to 40 mm.

10. The pressure relief pad preventing facial pressure ulcers as claimed in claim 8, wherein the cutout portion is cut and separated from the bottle body portion, so that the bottle body portion has a hollow portion; the bottleneck portion corresponds in position to the bridge of a user's nose, the bottle body portion covers the user's cheeks and philtrum and is attached tightly to the skin, and the user's nose is exposed out of the hollow portion; and the cutout portion is placed on the user's forehead and attached tightly to the skin.
